(19) United States
(12) Patent Application Publication
(10) Pub. No.: US 2014/0005281 A1
(43) Pub. Date: Jan. 2, 2014

(54) Method of Predicting Increased Risk of Suffering Statin-induced Adverse Drug Reactions

(71) Applicants: Jose A. Lasalde, SJ, PR (US); Orestes Quesada, (US); Carlos Baez, San Juan, PR (US); Gary Grajales, St. Louis, MO (US); Walter Silva, Humacao, PR (US)

(72) Inventors: Jose A. Lasalde, SJ, PR (US); Orestes Quesada, (US); Carlos Baez, San Juan, PR (US); Gary Grajales, St. Louis, MO (US); Walter Silva, Humacao, PR (US)

(21) Appl. No.: 13/540,598

(22) Filed: Jul. 2, 2012

(51) Int. Cl. C12Q 1/68; G01N 33/68

(52) U.S. Cl. CPC C12Q 1/6883; G01N 33/6887
USPC 514/789; 435/6.11; 435/7.21

(57) ABSTRACT

Inhibitors of 3-hydroxy-3-methylglutaryl-coenzyme A reductase (statins) are prescribed to lower serum cholesterol levels and reduce the risk of CVD. Despite the success of statins, many patients abandon treatment owing to neuromuscular adverse drug reactions (ADRs). Genome-wide association studies have identified the single-nucleotide polymorphism (SNP) rs4149056 in the SLCO1B1 gene as being associated with an increased risk for statin-induced ADRs.

By studying slow-channel syndrome transgenic mouse models, this invention determined that statins trigger ADRs in mice expressing the mutant allele of the rs137852808 SNP in the nicotinic acetylcholine receptor (nAChR) α-subunit gene CHRNA1. Mice expressing this allele show a remarkable contamination of end-plates with caveolin-1 and develop early signs of neuromuscular degeneration upon statin treatment. The invention demonstrates that genes coding for nAChR subunits may contain variants associated with statin-induced ADRs.

### GOVERNMENT INTEREST

The claimed invention was made with U.S. Government support under grant numbers 2R01GM56371-12, SNRP U54NSO430311 and R01NS033202 awarded by the US National Institutes of Health (NIH). The government has certain rights in this invention.

### BACKGROUND OF THE INVENTION

According to the World Health Organization (WHO), cardiovascular disease (CVD) is the world's leading cause of death. It has been estimated that 17.3 million people died from CVD in 2008 alone, representing 30% of all global deaths. High levels of cholesterol carried by low-density lipoprotein, colloquially known as ‘bad cholesterol’, is a firmly established independent risk factor for CVD. Naturally, as CVD claims so many lives, cholesterol-lowering medications represent an essential strategy to reduce CVD mortality rates. Inhibitors of 3-hydroxy-3-methylglutarylcoenzyme A reductase-collectively named statins-inhibit the rate-limiting step in the biosynthesis of cholesterol, thus reducing its availability and effectively lowering plasma low-density lipoprotein levels. Despite its demonstrated safety, a fraction of those treated with statins suffer adverse drug reactions (ADRs), mostly neuromuscular symptoms, which eventually force patients to discontinue treatment. Because the number of patients on statins is so high (more than 17 million people are prescribed Lipitor™), the actual number of patients that abandon treatment due to ADRs can be substantial. Many are left with an increased risk for CVD as there is a linear dose-response relationship between increasing adherence to statin treatment and decreasing coronary mortality.

The most common statin ADRs are neuromuscular problems involving muscle pain or weakness, and range in severity from myalgia to rhabdomyolysis. Myalgia is defined as muscle weakness or pain without an elevation in serum creatine kinase (CK) levels. In clinical trials, the incidence of myalgia is 1-5%, although observational studies reveal that myalgia is more frequent (9-20%) than expected. Very rare muscle-related ADRs include myositis, which refers to muscular symptoms with serum creatine kinase elevation, and the life-threatening rhabdomyolysis, which is characterized by a marked creatine kinase elevation and can cause severe muscle pain, renal failure, disseminated intravascular coagulation, and death. Genetic factors have been suspected to have a role in the etiology of ADRs, thus prompting genome-wide association studies (GWAS), and candidate gene studies looking for genes associated with increased risk for ADRs. The SLCO1B1 gene has been shown in several studies to be associated with statin ADRs, exceeding even the stringent P-value thresholds of GWAS. Candidate genes have been selected on the basis of their hypothesized role in the etiology of statin-induced ADRs, such as genes coding for proteins involved in pain perception, vascular homeostasis, statin transport into hepatocytes, and drug metabolism, among others. However, while neuromuscular problems are among the most common ADRs, genes coding for proteins expressed in the neuromuscular junction (NMJ) are absent in candidate gene studies.

The nicotinic acetylcholine receptor (nAChR) is a transmembrane glycoprotein highly expressed in skeletal muscle NMJs that transduces the chemical signal of acetylcholine released by nerve endings into an electrical signal and subsequent muscle contraction. Owing to its fundamental role in the transmission of nerve impulses across the NMJ, mutation-induced structural changes in nAChRs can substantially alter its function and affect nerve transmission across the synapse, resulting in muscle weakness and pain. For instance, slow-channel congenital myasthenic syndromes (SCS), characterized by generalized muscle weakness and fatigability, result from point mutations in nAChRs that extend channel open time. Previous experiments showed that nAChR mutations can also dramatically modify cholesterol-dependent regulation of receptor function. Therefore, it is believed that statin sensitivity is merely a manifestation of the cholesterol sensitivity of nAChR genetic variants.

### SUMMARY OF THE INVENTION

Using transgenic mouse models expressing different SCS mutations, we demonstrated that the αC418W mutation produced a myopathy-like picture upon statin treatment resembling statin-induced ADRs. The nonsynonymous single-nucleotide polymorphism (SNP) rs137852808 (αC418W), responsible for a mild SCS, was found to be cholesterol-sensitive, as its macroscopic response to agonist stimulus increased significantly upon cholesterol depletion. The present invention demonstrates that genetic variants of genes coding for nAChR subunits could be related to an increased risk for statin-induced ADRs, and suggest that detailed, statistically powered candidate gene studies, including the nAChR genes and perhaps other genes coding for proteins expressed in the NMJ, are likely to result in the identification of variants related to statin-induced ADRs and are therefore warranted.

### BRIEF DESCRIPTION OF THE DRAWINGS

Further features and advantages of the invention will become apparent from the following detailed description taken in conjunction with the accompanying figures showing illustrative embodiments of the invention, in which:

FIG. 1 shows a plot of cholesterol content vs. days during statin treatment according to the present invention.

FIG. 2 shows velocity vs. time plots for PBS-treated WT and αC418W mice for 36 days according to the present invention.

FIG. 3a-d shows a representation of the mice locomotor activity analysis according to the present invention.

FIG. 4 shows a plot for statin-treated αC418W mice and PBS-treated WT mice endplate size distribution according to the present invention.

FIG. 5 shows a plot of caspase-3 activity for WT, αV249F and 6S2621 mice according to the present invention.

FIG. 6a shows a plot of N-fold change in decrement for WT, αV249F, αC418W and δS2621 according to the present invention.

FIG. 6b shows representative recordings of αC418W CMAP according to the present invention.

FIG. 7 shows velocity vs. time plots for statin-treated WT and αC418W mice for 36 days according to the present invention.

FIG. 8a shows images of tibialis anterior muscles for WT and αC418W mice according to the present invention.

FIG. 8b shows a plot of GBHA-labeled αC418W endplates vs. days for PBS-treated and statin-treated αC418W mice according to the present invention.

FIG. 8c shows a plot of caspase-3 activity vs. days for PBS-treated and statin-treated αC418W mice according to the present invention.

FIG. 8d shows a plot of caspase-3 activity vs. days for PBS-treated and statin-treated WT mice according to the present invention.

FIG. 9a shows a plot of end-plate size distribution for PBS-treated and statin-treated WT mice according to the present invention.

FIG. 9b shows a plot of end-plate size distribution for PBS-treated and statin-treated αC418W mice according to the present invention.

FIG. 10a shows images for αC418W showing a significant amount of Cav-1 colocalizing with endplates according to the present invention.

FIG. 10b shows a plot of Cav-1 positive end-plate after days for PBS-treated and statin-treated αC418W and WT mice according to the present invention.

Throughout the figures, the same reference numbers and characters, unless otherwise stated, are used to denote like elements, components, portions or features of the illustrated embodiments. The subject invention will be described in detail in conjunction with the accompanying figures, in view of the illustrative embodiments.

### DETAILED DESCRIPTION OF THE INVENTION

### Materials and Methods

### Voluntary Wheel Running

The onset and progression of weakness in both animal models (αC418W and WT) was monitored to determine the effects of statin treatment. A computer-monitored mouse activity wheel system (wheel counter model 86061, wheel diameter 12.7 cm,

clear polycarbonate cage, USB computer interface model 86056A, activity wheel monitor software version 9.2, Lafayette Instruments, Lafayette) was used to determine exercise and locomotor activity profile of mice during treatment. This system monitored the average velocity of the activity wheel during 24 hours. The computer logged the average velocity (meters/minute) and the cumulative distance (meters) the mouse traveled for every second along the course of these 24 hours. Once this file was obtained, the file was opened in Excel™ and the entire A column was filtered to display the average velocity, since this was the variable that was going to be analyzed. After this, all the data contained in the file, except the average velocity, was erased so that only a column containing all the average velocities could be saved as a coma separated value (CSV) file. This file was then renamed from a .csv to a .dat file so that it could be analyzed in a custom made program. This program analyzes the moments in which the activity wheel's velocity was greater than 0 and calculates two values. First it calculates for how long (seconds) the mouse ran and second it calculates the average velocity of this activity period (meters/minute). Once these measurements were performed, the activity period duration and its corresponding average velocity were logged in side-by-side columns. This was organized so that the activity period duration is in an ascending order starting with the lowest value, which is always 1 second. Activity periods that exhibit the same duration were all displayed with their respective average velocity. The code for the custom program was written in the C⁺⁺ programming language and can be compiled in the Bloodshed Dev-C++ software to produce an executable (.exe) file that is the custom program per se. These values were then analyzed with Sigma Plot (Systat Software Inc., San Jose, Calif.), which divides a scatter plot generated with the aforementioned (activity interval duration and its respective average velocity) data into a 10×10 grid. Then, Sigma Plot calculated the frequency of the data points contained within each grid unit. The obtained frequency values were then used in MatLab™ 7.4 R2007a (The MathWorks™, Inc., El Segundo, Calif.) to produce contour plots. Velocity bin values were on the X-axis and time bin values (in log scale) are on the Y-axis, and the frequency information was displayed as colored contours. Increased frequencies were represented as a shift from blue to red contours. In order to display the dynamic range of the data, 15 contours were distributed following a cubic curve with a final contour level that displays a maximum frequency of 22. As such, the final contour (22) contains all data equal or greater than its threshold value. This arrangement allows for low frequency contours to be closer spaced than high frequency contours, providing more detail in the areas of the histogram representing mouse activity. Before starting the experiment mice were placed in a similar cage with a similar activity wheel so that they learned how to run prior to the first experiment day. Once the activity recording finished (24 hours) the mouse was returned to its original cage. In order to prepare the cage for a new animal, each cage and activity wheel was washed with tap water and cleaned with 70% alcohol after each experiment day; the bedding was also changed.

### Custom Made Program

/*
This program converts a list of continuous second-to-second velocities into a list of time intervals (delimited by sub threshold velocities) with their respective average velocities. It accepts as input a tab-delimited list of continuous time-points (in seconds) with an instantaneous velocity for every time point; the data must be in a *.dat file. The output is also a *.dat file.

### Animals, Care and Procedures

Male 6-8 weeks-old mice that express the αC418W mutation on the muscle nAChR, and WT (FVB) were used. FVB mice were used as controls for αC418W and αV249F while C57BL/6 was used for δS262T since these mutant mice were created using these respective background strains. All the stable transgenic mice have been inbred for vastly more than 15 generations, inheriting the transgene in a simple Mendelian fashion. Mutant transgenic mice were previously established and described in detail. All animals were bred and housed in an environmentally controlled facility (10/14-h light/dark cycle, temperature between 20-22° C., relative humidity 65-75%) and have free access to food (Harlan Laboratories, IN) and tap water. All protocols were approved by the University of Puerto Rico Institutional Animal Care and Use Committee (IACUC). To screen the transgenic mice, genomic DNA was recovered from mouse-tail tips using DNeasy kit (Qiagen) following manufacturer instructions. The presence of the transgene was determined by polymerase chain reaction (PCR) using PCR beads (GE) and primers to amplify α and δ subunit genes and the NEO gene. The NEO gene primer was used to identify every transgenic line since only these mice have the gene, and the second primer varied upon which subunit contained the mutation. PCR products were visualized in agarose gel electrophoresis. Upon completion of experiments, all animals were euthanized by cervical dislocation and disposed according to institutional policies.

### Statin Treatment

Freshly prepared Atorvastatin calcium (Lipitor®) (44 mg/kg) (5 mg/ml) or placebo (PBS, 1×) was administered intragastrically via oral gavage with a metal feeding tube (Popper & Sons, Inc., NY) daily up to 36 days.

### Electromyography

Evoked compound muscle action potential (CMAP) responses were recorded in mice weighting 20-30 g using a Dual Bio Amp/Stimulator coupled to a Power Lab 4/30 data acquisition system (ADInstruments, CO) under Avertin anesthesia as described by the prior art. The CMAP responses were generated by the sciatic nerve stimulation. In order to do this an incision lateral and parallel to the femur was performed. This incision exposed the sciatic nerve, to which a copper wire was encircled. After this, the copper wire was coupled to an electrode that delivered a train pulse of 10 stimuli at a frequency of 5 Hz during of 0.05 ms. The percentage of decrease in amplitude (mV) of the CMAP (decrement) was calculated using the amplitude (peak positive to peak negative) of the 1^{st} and 10^{th} responses.

### Confocal Microscopy Imaging

For the NMJ size measurement, images were collected in the Confocal Imaging Facility at the University of Puerto Rico (CIF-UPR) using a Zeiss LSM 510 Laser Scanning Confocal Microscope (Carl Zeiss, Inc.). Endplates were labeled by incubating in Alexa-Fluor® 488-conjugated α-bungarotoxin (Invitrogen) for 1 hour and washed 3 times with PBS1X (15 min). Motor endplates were visualized using a 40× objective. Zeiss LSM 510 parameters were optimized at the beginning of every tissue sample observation. In order to obtain a good representation of the endplate population, the hemidiaphragms were divided into 5 sections, dividing the space between the ventral and dorsal part of the hemidiaphragm equally. Once all the images were obtained, 10 Z-stacks were acquired per mouse. Each one of these sections was imaged with the aforementioned parameters. Collected Zstacks were analyzed using the Imaris x64 6.1.3 software (Bitplane Inc., CT) in which a surface was generated over the reconstructed endplates so that its size could be calculated in three dimensions. These measurements were then plotted as normalized histograms so that changes in the sample distribution could be observed. These histograms were fitted using Peakfit (Systat Software Inc., CA). In order to perform the caveolin-1 (Cav-1) staining the tibialis anterior muscle was used. Once dissected, it was rapidly dipped in 2-methyl-butane (Sigma-Aldrich) bathed by liquid nitrogen. Once frozen, tissues were mounted in OCT compound so that the muscle could be cut in 10 μm slices using a cryostat (Leica, model CM1100, Leica, IL). Then, tissues were fan dried for 20 minutes and immersed into an acetone-methanol (1:1) mixture for 20 minutes at −20° C. Following fixation, tissues were fan dried once again for 20 minutes. In order to block the tissues, muscle slices were immersed in blocking solution (2% NGS, 0.2% Triton X-100, 1% DMSO in PBS 1X) for 1 hour. To prepare the slides for the antibody addition a circular area was drawn around the tissue slice with a PAP pen, which creates a thin-filmed hydrophobic barrier that keeps the antibody solution localized. Once the hydrophobic film dried, the antibody solution (caveolin-1 antibody H-97, Santa Cruz Biotechnologies, diluted 1:500 in blocking solution) was added for 12-16 hours at 4° C. In order to wash the primary antibody, the tissue was immersed in washing buffer (0.05% Tween-20 in PBS 1X) 3 times for 10 minutes each. Finally, the secondary antibody (Molecular Probes, goat anti rabbit 1:1000) was added for one hour at 25° C., and as before the tissue was washed 3 times 10 minutes each. Later, mounting medium for fluorescence with DAPI (H-1200, Vector Laboratories Inc.) was added. All Cav-1 imaging was performed in a TCS laser-scanning microscope (Leica, Ill.). The percentages of Cav-1 positive endplates were measured in order to compare the effects of the statin treatment and the difference on Cav-1 positive endplates between WT and αC418W mice.

Glyoxal-bis (2-hydroxyanil) Stain (GBHA)

GBHA histochemical staining was performed according to previously published methods. In brief, the tibialis anterior muscle was frozen in 2-methyl-butane bathed by liquid nitrogen followed by mounting in OCT compound and sliced in a cryostat at a 10 μm thickness, stained and mounted. Each slice was stained in the following order: slice #1 (cholinesterase stain), slice #2 (GBHA, calcium stain), slice #3 (cholinesterase stain). The slice (1 or 3) that exhibits the highest endplate number was selected and compared against slice #2 (GBHA-stained). Cholinesterase was stained by immersing the slices in a modified Ringer's solution (0.1% CuSO4.5H20, 0.2% glycine and 5 mM acetylcholine iodide adjusted to pH 6.5 with a few drops of a 10% solution of 2-amino-2-metylpropa-1-ol). After 15 minutes in Ringer's solution at room temperature the slices were rinsed in distilled water and placed in a 1% solution of yellow ammonium sulfide (pH 9) for 5 seconds, followed by distilled water rinse and subsequent immersion in ethanol 70%. The calcium stain was prepared by mixing 16 ml 0.4% glycoxal bis-2-hydroxyanil dissolved in methanol with 7.2 ml NaOH 5%. Then, slides were immersed in this solution and air dried for 2 minutes followed by additional immersion and air dry for two minutes to finally rinse in 70% ethanol. After this, the slides were dipped in 0.25% methylene blue dissolved in 70% ethanol. The counterstained sections were dehydrated in acetone, cleared in xylene, and mounted.

### Caspase-3 Activity Experiments

A firefly luciferase-based assay was used to measure activity of caspase 3 (Caspase-Glo® 3/7, Promega). Muscles were homogenized (25 mM HEPES pH 7.5, 0.1% (v/v) Triton X-100, 5 mM MgCl2, 2 mM 1,4-dithiothreitol, 10 mM NH4Cl, 10 mM 3-methyladenine, 74 μM antipain, 0.15 μM aprotinin, 1.3 mM EDTA, 20 μM leupeptin, and 15 μM pepstatin). After homogenization, a 20 μg protein product was added to the luminometer in triplicates for the protease luminescence assay.

### Cholesterol Measurement in Muscle

800 μl of each sucrose gradient fraction were subjected to the Bligh-Dyer method for the extraction of lipids in solution. Briefly, 3.75 ml 1:2 (v/v) CHC13: MeOH were added to each sample, followed by 1.25 ml of CHCl3, and 1.25 ml of distilled water; after each addition, samples were vigorously vortexed. The organic phase of each sample was carefully extracted and dried under N2(g). Cholesterol was separated from other lipids on rhodamine 6G stained silica gel G plates with petroleum ether/diethyl ether (98:2, v/v) as the solvent system. The spots corresponding to cholesterol was extracted with petroleum ether:ethyl ether (2:3 v/v) and further assayed using the Wako cholesterol E Kit (Wako Chemicals USA, VA) according to the manufacturer's indications.

### Statistical Analysis

All experiments were replicated at least three times, with the number of replicates (n) indicated in the figure legend. Each replicate represents a mouse, and each data point was the average of at least three different samples. Bars in all figures represent the standard error of the mean (SEM). T-tests were performed using GraphPad Prism version 4.00 for Windows, GraphPad Software, San Diego, Calif., USA.

### RESULTS

αC418W Neuromuscular Transmission is Significantly Impaired after 3 Days of Statin Treatment

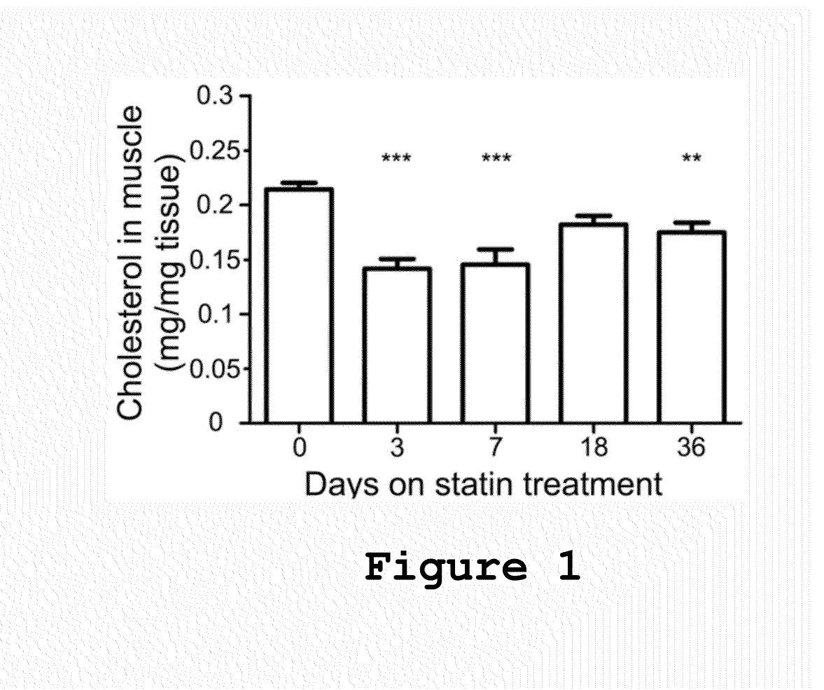

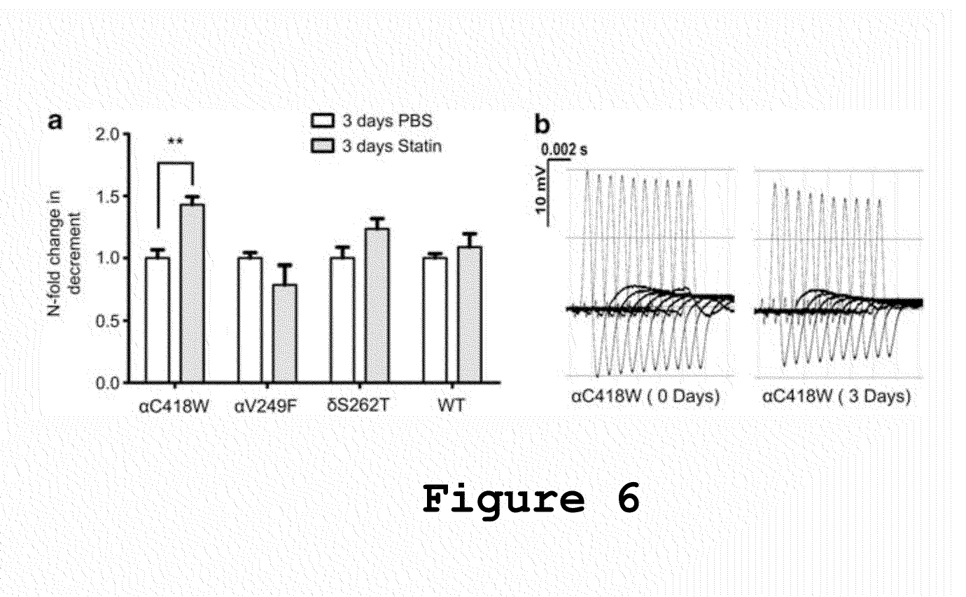

To screen for statin ADRs, three different transgenic lines expressing SCS mutations (αC418W, δS262T, and αV249F) for impairment in neuromuscular transmission were studied using repetitive stimulation electromyograms. This technique compares the amplitude of a series of compound muscle action potentials (CMAP) repetitively evoked by sciatic nerve stimulation for evidence of decrement in the CMAP, given that the decremental response is a typical pattern of myasthenic disorders. After 3 days of statin treatment, which significantly lowered total cholesterol levels as shown in FIG. 1, the αC418W compound muscle action potential amplitude decrement increased significantly (1.497±0.136 fold, P<0.01, n=4). In contrast, the statin treatment had no significant effect on the wild type (WT), δS262T, and αV249F mice as shown in FIG. 6(a).

Statin Treatment Induces Decreased Locomotor Activity in αC418W Mice after 18 Days of Atorvastatin Treatment

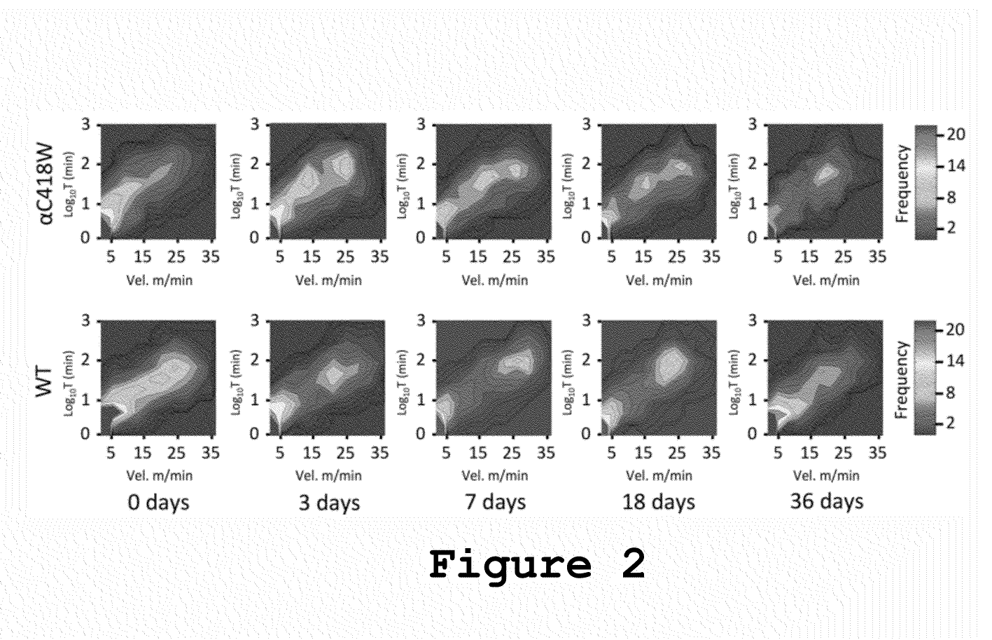

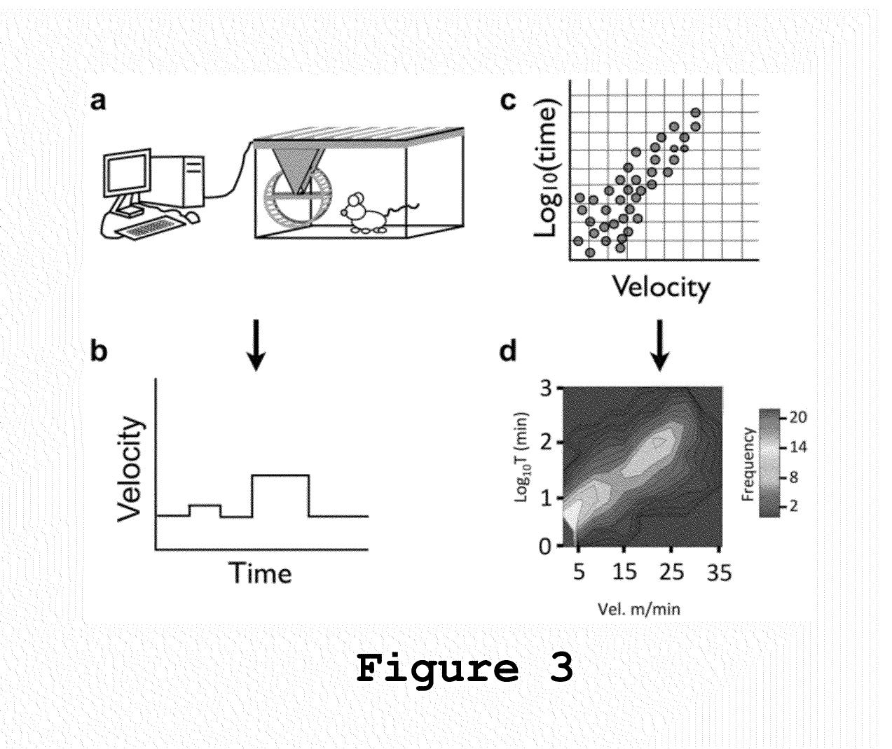

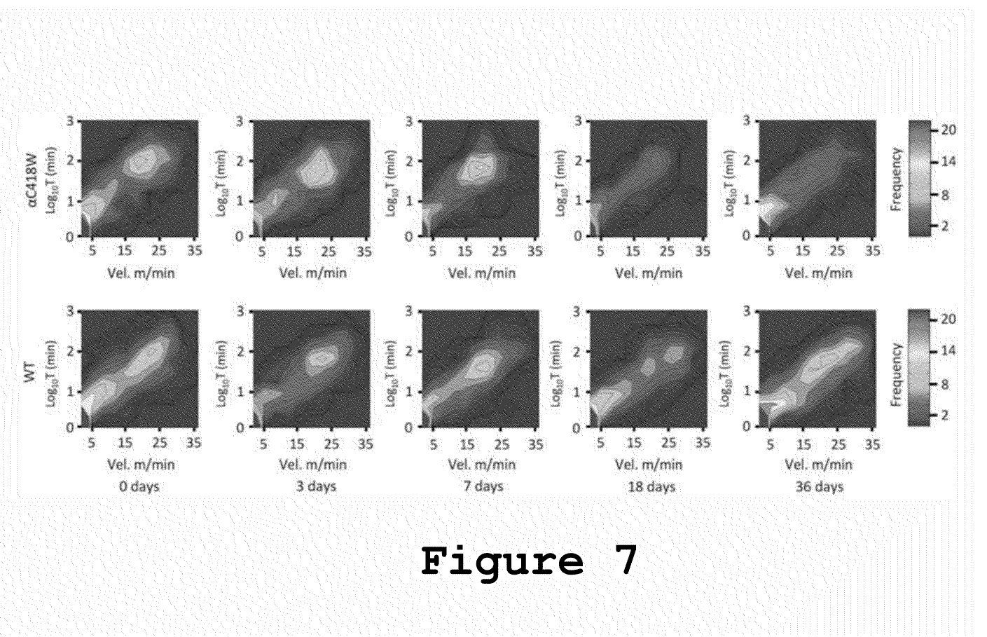

To determine whether the impairment in neuromuscular transmission in αC418W mice is associated with a change in physical activity levels, voluntary locomotor activity was measured during a 24-h period in αC418W and WT mice. We assessed the ability of mice to run for prolonged periods of time in a cage containing an activity wheel that recorded the instantaneous velocity at a frequency of 1 Hz over a 24-h period, FIG. 3(a). To understand the progression of ADR as a function of time, the activity measurements were obtained over a 36-day period at 3, 7, 18 and 36 days and the results plotted in heat maps as shown in FIG. 7. The most frequent activity is seen at low values of both velocity (X-axis) and duration (Y-axis), because this represents the periods when the mice ran for short intervals of time at low velocities. A second peak of activity occurs when the values of both velocity and duration are high. Because the high velocity- or long-duration peak represents periods when the mice are engaged in considerable physical activity, it is of particular interest. The initial differences between the WT and αC418W strains are negligible, but over the course of the statin treatment, the high velocity or long duration peak in αC418W is observed to progressively decrease in frequency, particularly at 18 and 36 days, in αC418W mice. These results suggest that αC418W mice are engaging in some physical activity, but it is being abandoned after short periods of time, causing a decrease in the frequency of higher-activity periods. WT animals treated over the same period with atorvastatin (FIG. 7) as well as placebo-treated αC418W and WT mice remained unchanged as shown in FIG. 2. This is consistent with a higher predisposition to suffer from statininduced ADRs.

### Statin-Induced NMJ Calcium Overload and Caspase Activation

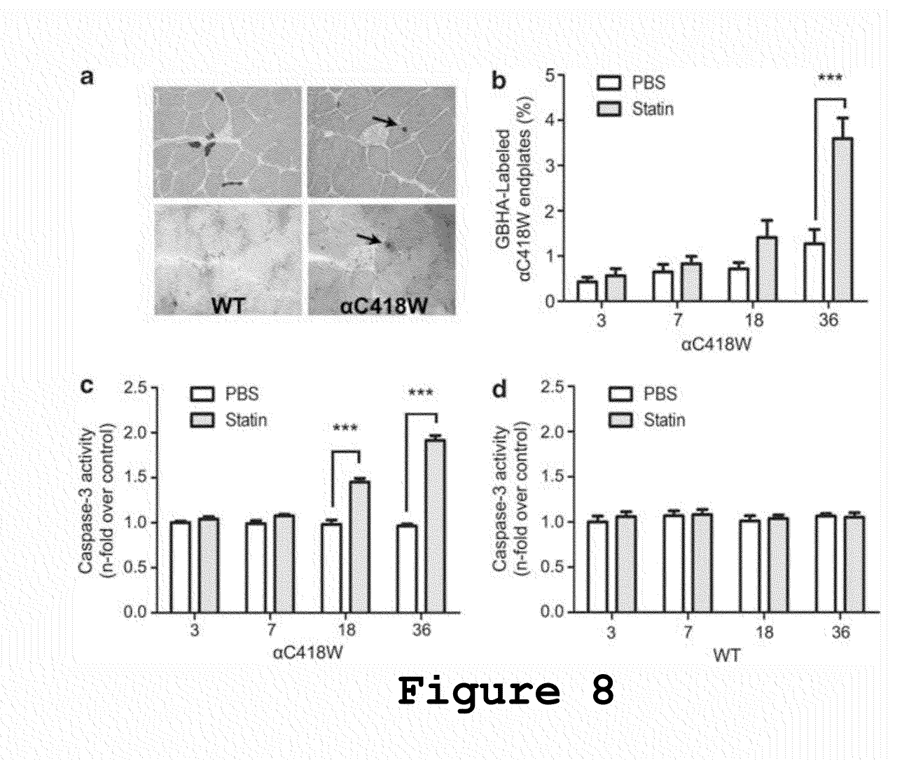

To further explore the effects of cholesterol sensitivity associated with the rs137852808 SNP, we performed histochemical studies to look for an effect on calcium overload of end-plates. Unlike other SCS mice and SCS patients, motor end-plates in αC418W mice had a low level of calcium overload. Serial cryosections of tibialis anterior muscle from statin-treated αC418W and WT mice were stained for calcium deposits (via GBHA) and also for cholinesterase to localize end-plates. Looking at these in adjacent sections, we were able to measure the percent of calcium-positive NMJs as previously described. In statin-treated αC418W mice, this calcium overload was observed to increase as a function of time, reaching nearly a 3-fold increase over control levels at 36 days (FIG. 8b). The statin treatment did not cause NMJ calcium overload in WT mice (FIG. 8a). We previously showed that increased end-plate calcium in the muscle of SCS patients and mice is associated with increased levels of activated caspases, proteases known to mediate apoptosis. To test for similar increases resulting from statin-induced end-plate calcium overload, we analyzed muscle homogenates using a luminometric assay for caspase-3, the downstream mediator of apoptosis, after 3, 7, 18, and 36 days of statin treatment. In αC418W mice, after 18 and 36 days of treatment, caspase-3 activity was increased 1.475- and 1.986-fold, respectively, as compared to controls (FIG. 8c), (P<0.001, n=5). As expected, in statin-treated WT mice, caspase-3 activity remained unchanged when compared with the placebo-treated control group. These findings of increased caspase-3 activity in statin-treated αC418W mice are consistent with the previous electrophysiological, behavioral, and histological findings.

The Distribution of End-Plate Size is Altered in Transgenic Mice after 36 Days of Statin Treatment

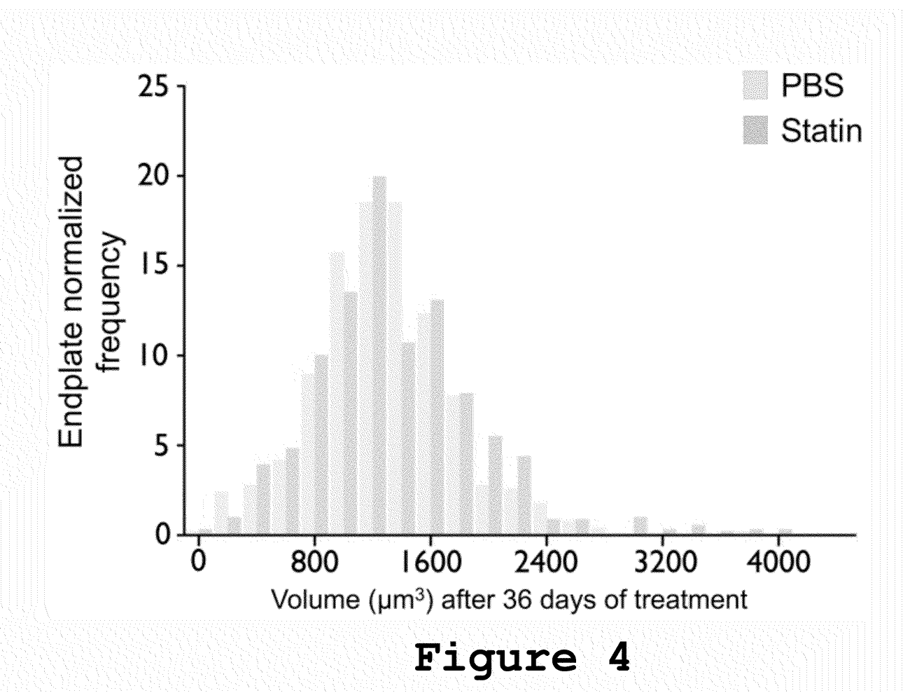

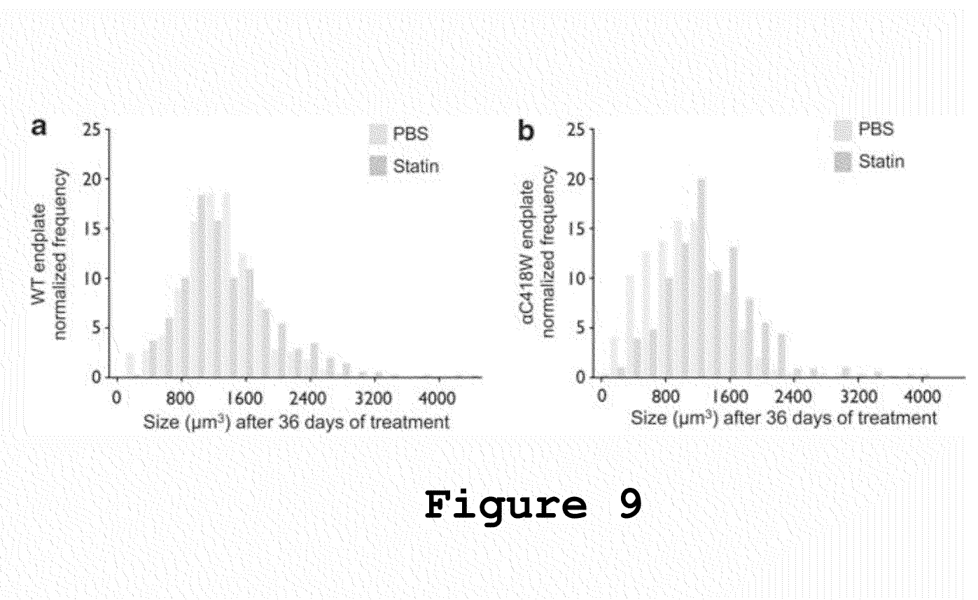

SCS is associated with distinct changes in end-plate morphology including simplification and shrinkage of end-plates, a finding that has proven to be reproducible in SCS mice. To investigate the effect of statin treatment on NMJ structure in the αC418W mice, we measured NMJ size using confocal fluorescence microscopy after labeling nAChRs with Alexa Fluor 488-conjugated abungarotoxin. This was done at day 36 of treatment, the time at which a maximum effect on locomotor-activity loss, calcium overload, and increased caspase activity was seen. After 36 days of statin treatment, WT NMJ size distribution remained unchanged (FIG. 9a). PBS treatment had no effect on either WT or αC418W end-plates. However, statin treatment caused an appreciable change in the size distribution of αC418W NMJs (FIG. 9b); it should be noted that the resulting distribution of statin-treated αC418W NMJs was not dissimilar from that seen in PBS-treated WT mice as shown in FIG. 4.

### Cav-1-Positive NMJs are More Numerous and Sensitive to Statins in αC418W Mice

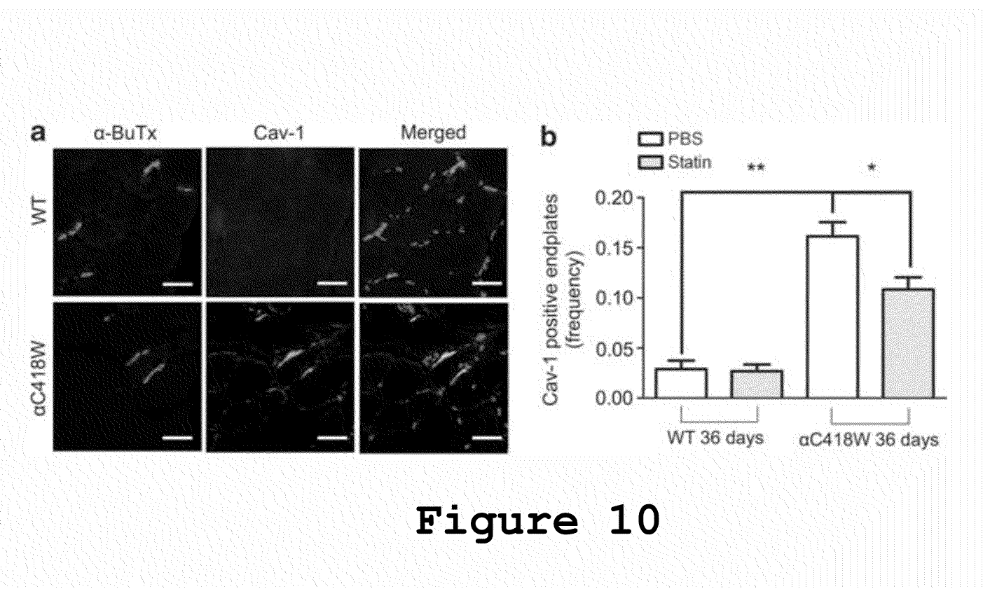

As previously reported, the αC418W mutation creates a caveolin-binding motif in nAChRs expressed in vitro. Immunohistochemistry was used to test the effect of the αC418W-caveolin binding motif on the distribution of Cav-1 by comparing the percentage of Cav-1-positive end-plates along the tibialis anterior muscle sections of αC418W and WT mice. After measuring the percentage of Cav-1-positive NMJs, we found increased immunolabeling of NMJs with anti-Cav-1 antibody in αC418W mice relative to WT mice (16.14±1.41% versus 2.90±0.83%, P<0.01, n=3), as shown in FIG. 10a, suggesting that this mutation increases the presence of Cav-1 within the αC418W NMJ. Finally, we explored the effect of 36 days of statin treatment on Cav-1 localization in αC418W mice. We found that the percentage of Cav-1 positive NMJs in αC418W mice was significantly reduced in statin-treated αC418W mice (from 16.14±1.41% to 10.84±1.21%, P<0.05, n=3), whereas the WT mice remained unchanged (from 2.90±0.83% to 2.69±0.66%), as shown in FIG. 10b, suggesting that retention of Cav-1 in αC418W endplates is sensitive to cholesterol concentration.

### DISCUSSION

CVD is the leading cause of death and a major cause of disability. High cholesterol levels are a major risk factor in the development of CVD. Medications that reduce cholesterol levels, such as statins, have been shown to decrease the incidence of cardiovascular events by 20-30% per mmol 1⁻¹ reduction in low-density lipoprotein. However, although proper use of statins may substantially decrease the likelihood of suffering from CVD, many at risk choose not to continue treatment. Indeed, nonadherence can be as high as 75% after 5 years of treatment. A significant reason for nonadherence to statins is the fear of suffering ADRs. Despite great efforts, the causes of statin-induced ADRs have remained elusive and unpredictable, causing excessive concerns among patients and leading to nonadherence. Unfortunately, nonadherence may be more dangerous than ADRs. For instance, nonadherence is associated with an 85% increase in mortality. We provide evidence that some ADRs may be related to genetic variants of membrane proteins, such as the nAChR, that result in an abnormal level of cholesterol sensitivity that affects the NMJ.

Genetic factors have been suspected to have a role in the etiology of ADRs, prompting GWAS and candidate gene studies to look for genes associated with increased risk for ADRs. GWAS, which seek to find common genetic variants statistically more prevalent in patients affected by disease or ADRs, have been demonstrated to work. However, because GWAS examine the whole genome, true signals can be overshadowed with statistical noise from variants not associated with ADRs. The P-value threshold to reach ‘genome-wide significance’ is therefore very low (5×10⁻⁸) to avoid false positives, and sample sizes are often in the thousands in order to have adequate power to detect associations. GWAS necessary stringent P-values also mean that potentially important genetic variants may not reach genomewide significance if the sample size is not large enough. By focusing on a small number of genes rather than examining the whole genome, the candidate gene approach may have a higher statistical power, identifying genes as associated with risk for ADRs in studies with smaller sample sizes. In addition, rare variants of individually large effect can be identified in candidate gene-resequencing studies. However, the candidate gene approach is hypothesis-driven, and thus limited by how much is known about the underlying biology of the disease mechanism. The underlying mechanism for statin ADRs is not completely understood, thus the selection of candidate genes can be challenging. Genes selected as candidates on the basis of their hypothesized role in the etiology of statin-induced ADRs include genes encoding the organic anion-transporter polypeptide member 1B1, which is expressed in the hepatocyte basolateral membrane and is responsible for the hepatocellular uptake of endogenous and foreign substances, including statins; serotonin receptors and transporters involved in pain perception; angiotensin II Type 1 receptors and nitric oxide synthase 3, which are involved in vascular homeostasis; and cytochrome P450 drug metabolizing enzymes, among other proteins. Nevertheless, although neuromuscular problems are among the most common ADRs, candidate gene studies focusing on genes coding for proteins expressed in the NMJ are lacking.

The nAChR has a pivotal role in neuromuscular transmission. Indeed, point mutations in the four subunits making up the nAChR are responsible for SCS, which are disorders of neuromuscular transmission characterized by muscle weakness and fatigability. To study the potential role of the nAChR in the etiology of statin-induced ADRs, we screened three different transgenic animals expressing SCS-causing mutations-including the cholesterol-sensitive αC418W mouse-for impaired neuromuscular transmission upon atorvastatin treatment by means of EMG experiments. We hypothesized that, among the SCS transgenic mice studied, only the αC418W mouse would display impaired neuromuscular transmission following a drop in membrane cholesterol concentration achieved through statin treatment, consistent with previous studies that established the cholesterol-sensitive nature of the αC418W nAChR. As hypothesized, the transgenic mouse model expressing the cholesterol-sensitive allele of the nonsynonymous SNP rs137852808 (αC418W) was the only strain that developed impaired neuromuscular transmission upon atorvastatin treatment as shown in FIG. 6a

To further characterize the atorvastatin sensitivity displayed by this nAChR genetic variant, we devised a novel technique to study the voluntary locomotor activity of these mice using a running wheel. This technique permitted us to measure the frequency at which the mice ran for a specific period of time and at a specific velocity. Using this method, we demonstrated that atorvastatin treatment transforms otherwise seemingly normal mice into weakened mice, as determined by their unwillingness or inability to run at relatively high velocities for long periods of time (FIG. 7).

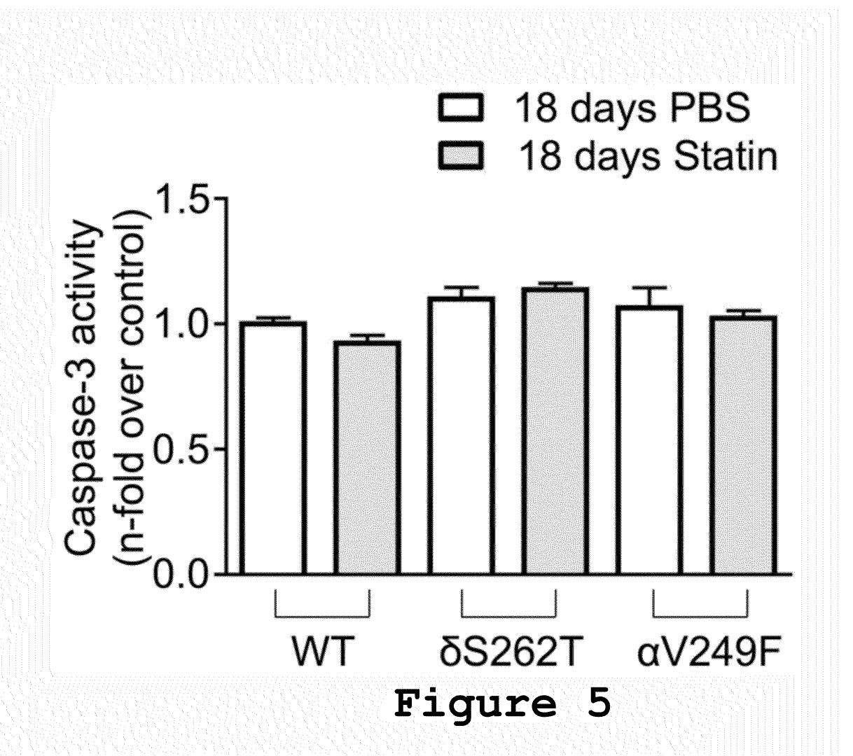

Previous studies have shown that SCS mutant mice, including αC418W mice, develop calcium overload of NMJs. Here we found that upon prolonged atorvastatin treatment, the proportion of NMJs overloaded with calcium in αC418W transgenic mice increased when compared with the placebo-treated αC418W mice (FIG. 8b). In contrast, statin treatment did not cause calcium overload at any NMJ in WT mice (FIG. 8a). The activity of mutant nAChRs and local disturbance of the ionic milieu, such as calcium overload, have been presumed to result in the eventual activation of caspases. As expected, examination of caspase-3 activity showed that αC418W mice had increased levels at 18 and 36 days of statin treatment, whereas WT mice showed no response. This phenomenon was exclusive to αC418W mice, as δS262T, and αV249F mice did not show an increase in caspase-3 activity as shown in FIG. 5.

We used confocal microscopy to examine transgenic mice endplates, fluorescently labeled with Alexa Fluor 488-conjugated abungarotoxin, to gain insight into the effects of atorvastatin treatment on end-plate integrity. Our results demonstrate that the distribution of end-plate size is altered in the αC418W mice upon statin treatment but not in WT mice. In addition, careful examination revealed that upon 36 days of atorvastatin treatment, the size distribution of transgenic mice end-plates overlapped the distribution of WT mice end-plates as shown in FIG. 4. Our transgenic mice were created by microinjection of single-cell mouse embryos and the expression of the transgene was highly variable among fibers. Such variations may affect the proportion of mutant nAChRs in a given end-plate. In light of this, our results suggest that the proportion of end-plates with a higher expression of the nAChR transgene is reduced upon statin treatment. These end-plates may be more susceptible to the concomitant cholesterol depletion of statin treatment, which is consistent with the previously reported cholesterol sensitivity of the αC418W nAChR.

We found that there was a significantly higher proportion of Cav-1-positive end-plates in αC418W mice compared with WT mice. Upon 36 days of statin treatment, the proportion of Cav-1 in αC418W mice end-plates was significantly reduced (FIG. 10b). Caveolins are structural proteins that are indispensable components of the cholesterol-rich membrane raft domains, known as caveolae, and exist in three isoforms, namely Cav-1, Cav-2 and Cav-3. Cav-3 is generally regarded as the caveolin isoform expressed in muscle; therefore, the unexpected presence of Cav-1 in the transgenic mice end-plates may be associated with sensitivity to atorvastatin treatment. Caveolins bind cholesterol in a 1:1 ratio, and thus its expression in end plates could be increasing their cholesterol levels. Presumably, contamination of end-plates with Cav-1 (and Cav-1-positive membrane domains), which are very dependent on cholesterol concentration, confers upon αC418W-expressing end-plates, a susceptibility to cholesterol reductions, a phenomenon that is not present in WT end-plates. This contamination could contribute to changes in end-plate plasticity and have a prominent role in the etiology of the concomitant statin-induced ADRs that lead to end-plate myopathy.

This invention demonstrates that genetic variants of the nAChR could be related to the onset of statin-induced ADRs. This is exemplified by the nonsynonymous SNP rs137852808 of the α subunit of the nAChR (αC418W), which showed a remarkable increase in risk for suffering an atorvastatin-induced ADR. The mechanism appears to relate to an effect of the variant rendering the NMJ sensitive to changes in cholesterol and, therefore, the action of statins. An alternative hypothesis could be that the prolonged gating kinetics of αC418W, rather than its cholesterol-sensitive nature, renders it sensitive to blockade by atorvastatin and hence produces an increased risk for statin-induced ADRs. This possibility is unlikely based on the finding that other slow-channel transgenic mice expressing δS262T, and αV249F mutations have no impairment of synaptic transmission or caspase activation after atorvastatin treatment (FIG. 5). These experiments demonstrate that slow channels are not necessarily risk factors for statin-induced ADRs. Clinical trials have demonstrated that statin-induced ADRs affect a relatively modest fraction of those who were prescribed the medication; however, in clinical practice the incidence of ADRs is greater than in controlled trials. Furthermore, the prevalence of statin-induced ADRs dramatically increases to 25% in patients who exercise and to >75% in professional athletes. This raises the paradoxical situation where exercise appears to be contraindicated in statin-treated patients. Nevertheless, despite the inherent dangers of statin-induced ADRs, the real danger is the discontinuation of statin treatment, as adherence may be a matter of life or death for CVD patients. In a recent survey, a 26% rate of nonadherence in patients with coronary artery disease was associated with an alarming 85% increase in overall mortality.

Although the present invention has been described herein with reference to the foregoing exemplary embodiment, this embodiment does not serve to limit the scope of the present invention. Accordingly, those skilled in the art to which the present invention pertains will appreciate that various modifications are possible, without departing from the technical spirit of the present invention.

We claim:

1-14. (canceled)

15. A method for predicting increased risk of suffering statin-induced adverse drug reactions comprising:
detecting genetic variants of genes coding for proteins expressed in the neuromuscular junction.

16. The method of claim 15, wherein said genes comprise a nicotinic acetylcholine receptor.

17. The method of claim 15, wherein said genetic variant comprises a single-nucleotide polymorphism rs137852808.

18. The method of claim 15, wherein said genetic variants result in the introduction of a caveolin binding motif in said proteins expressed in the neuromuscular junction.

19. The method of claim 15, wherein said genetic variants result in the introduction of a caveolin binding motif in a nicotinic acetylcholine receptor expressed in the neuromuscular junction.

20. The method of claim 15 comprising: detecting the presence of genetic variant rs137852808 that result in the introduction of caveolin binding motif in the nicotinic acetylcholine receptor expressed in the neuromuscular junction.

21. A method for predicting increased risk of suffering statin induced adverse drug reactions comprising:
detecting the protein caveolin-1 in the neuromuscular junction.

22. The method of claim 21, wherein said protein caveolin-1 in the neuromuscular junction is detected by immunofluorescence.

23. The method of claim 21, wherein said protein caveolin-1 in the neuromuscular junction is detected by western blot.

24. A method for treating statin-induced adverse drug reactions comprising: stabilizing calcium concentrations in the neuromuscular junction.

25. The method of claim 24, wherein the calcium concentrations in the neuromuscular junction are stabilized by pharmacotherapeutics.

26. The method of claim 24 comprising: providing ion channel blockers of calcium-permeable proteins expressed in the neuromuscular junction.

27. The method of claim 24 comprising: providing ion channel blockers of the nicotinic acetylcholine receptors expressed in the neuromuscular junction.

28. The method of claim 24 comprising: inhibiting inositol triphosphate receptors.
